(19) United States
(12) Patent Application Publication
(10) Pub. No.: US 2014/0135198 A1
(43) Pub. Date: May 15, 2014

(54) DEVICES AND METHODS FOR NUCLEIC ACID EXTRACTION CAPTURE AND CONCENTRATION USING ELECTRIC FIELDS AND CENTRIFUGATION

(71) Applicant: CALIFORNIA INSTITUTE OF TECHNOLOGY, PASADENA, CA (US)

(72) Inventors: Aditya RAJAGOPAL, IRVINE, CA (US); Carl J. ALLENDORPH, LOS ANGELES, CA (US); Axel SCHERER, BARNARD, VT (US)

(73) Assignee: CALIFORNIA INSTITUTE OF TECHNOLOGY, PASADENA, CA (US)

(21) Appl. No.: 14/062,763

(22) Filed: Oct. 24, 2013

Related U.S. Application Data
(60) Provisional application No. 61/725,390, filed on Nov. 12, 2012.

(51) Int. Cl. C12M 1/00

(52) U.S. Cl. CPC C12M 47/06
USPC 494/36; 494/50; 494/51; 494/37

(57) ABSTRACT

Methods and devices for molecular analysis are disclosed, based on centrifugation. A centrifuge device has centrifuge tubes and elements to create electric fields. The shear forces applied to the cells inside a solution with biological molecules permit the performance of different analytic techniques, such as lysis and sample preparation for PCR.

### CROSS REFERENCE TO RELATED APPLICATIONS

The present application claims priority to U.S. Provisional Patent Application No. 61/725,390, filed on Nov. 12, 2012, and may be related to U.S. patent application Ser. No. 14/048,735, filed on Oct. 8, 2013, the disclosure of both of which is incorporated herein by reference in their entirety.

### TECHNICAL FIELD

The present disclosure relates to biomolecular analysis. More particularly, it relates to devices and methods for nucleic acid extraction, capture and concentration using electric fields and centrifugation.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 illustrates a cross-sectional view of part of an exemplary magnetic field lysis and electric field nucleic acid capture device.

FIG. 2 illustrates an exemplary top-down view of magnetic dipoles and electrical dipoles for magnetic lysis and nucleic electrophoretic nucleic acid capture.

FIG. 3 illustrates an example of a two-part centrifuge tube.

FIG. 4 illustrates an exemplary tube with electrodes and no beads.

FIG. 5 illustrates an exemplary electrode arrangement for a centrifuge layout.

FIG. 6 illustrates a top view of another exemplary tube arrangement.

### SUMMARY

In a first aspect of the disclosure, a centrifuge device is described, the centrifuge device comprising: a rotating head; at least one centrifuge tube; at least one slot in the rotating head, configured to accept the at least one centrifuge tube; and at least one field element in the rotating head, wherein at least one field element is configured to generate an electric field.

### DETAILED DESCRIPTION

The polymerase chain reaction (PCR) is a critical technique in the detection and amplification of nucleic acid products. However, before PCR can be performed, DNA must be liberated and purified from serological samples. While there are chemical kits that can be used to perform both lysis and nucleic acid purification, such methods require significant time-intensive, and highly-skilled technical labor to implement. The present disclosure describes several automated instruments and procedures that perform these tasks in a way that results in significant cost- and time-savings. As known to the person skilled in the art, lysis comprises breaking down the cell walls or membranes, thereby causing the liberation of intracellular molecules.

Specifically, devices and methods based on centrifugation are disclosed. Cell lysis in centrifuges can be achieved by magnetically motivated shear. By accelerating and bashing magnetic particles against non-magnetic particles, or other granular material, in a centrifuge tube, it is possible to generate the shear forces necessary to tear cell walls and cell membranes. After cell lysis is performed in tin a centrifuge tube, orthogonal electric fields can be used for electrophoretic nucleic acid capture.

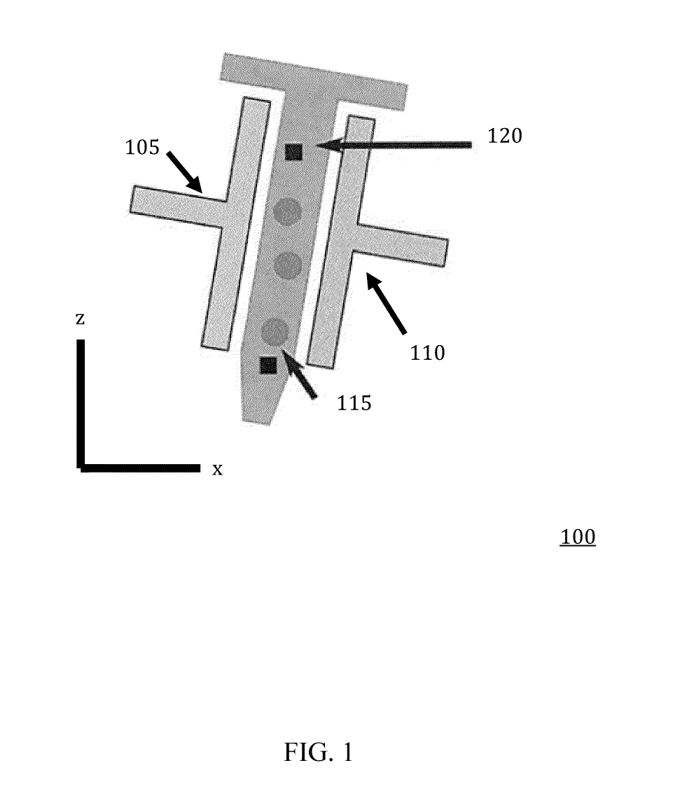

FIG. 1 illustrates a cross-sectional view of part of an exemplary magnetic field lysis and electric field nucleic acid capture device (100). The device (100) comprises a positive electrical electrode (105), a negative electrical electrode (110), non-magnetic beads (115), and magnetic particles (120).

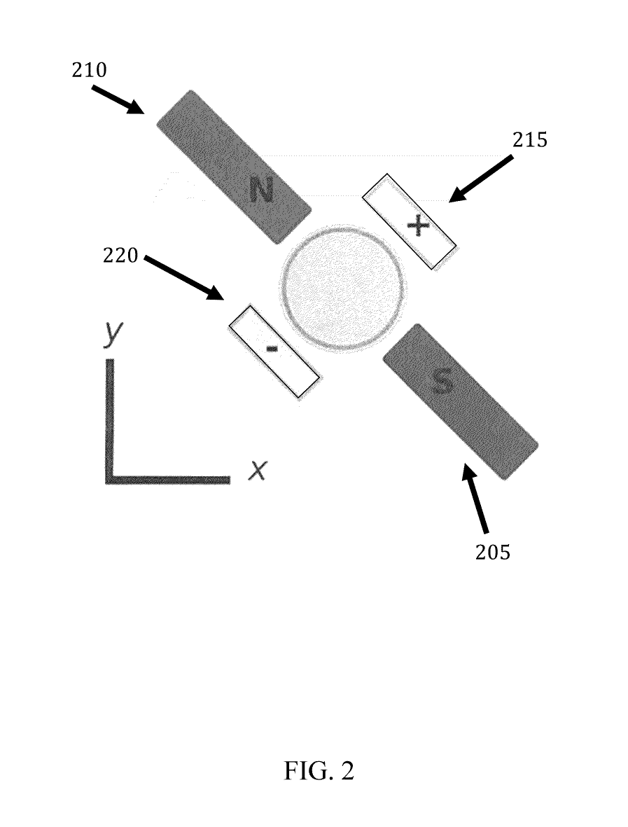

FIG. 2 illustrates an exemplary top-down view of magnetic dipoles and electrical dipoles for magnetic lysis and nucleic electrophoretic nucleic acid capture.

Referring to FIG. 2, a magnetic field may be formed by a south pole (205) and a north pole (210). The magnetic field may be formed, for example, by permanent magnets, or by electromagnets. An electric field may be formed by a positive pole (215) and a negative pole (220).

By coordinated control of the rotation velocity of the centrifuge, as well as of the frequency and magnitude of the electric field, nucleic acids can be selectively captured on the sidewalls of a tube.

For example, applying a time-varying electric field between a positive and a negative electrode in a centrifuge (such as elements 105 and 110 in FIG. 1), nucleic acids can be captured by one electrode, while supernatant and ionic solutions are washed to the bottom of the centrifuge tube.

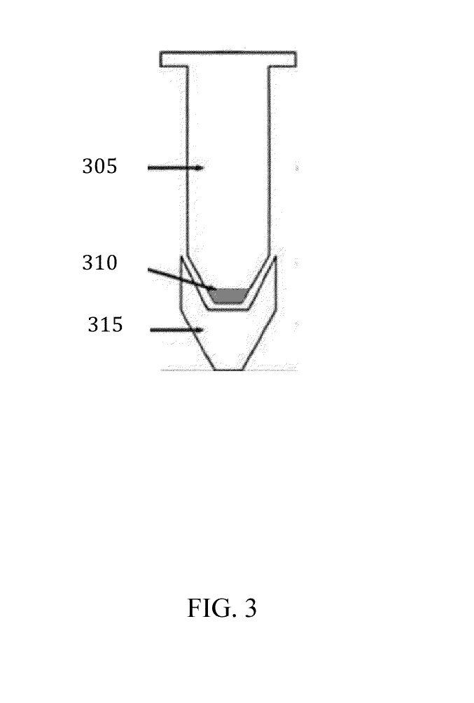

FIG. 3 illustrates an example of a two-part centrifuge tube. For example, a two-stage PCR centrifuge tube can comprise a main sample chamber (305), a filter and valve element (310), and a supernatant disposal bottom part (315).

By performing the centrifugation with a two part centrifuge tube (such as the tube in FIG. 3), the supernatant can be flowed into a waste receptacle (315), while the nucleic acid targets can be sequestered in the main centrifuge chamber (305). Controlling the rotational velocity of the centrifuge can allow for controllable flow rate past a selective filtered valve (310). By carefully controlling the flow rate, optimal conditions for cell lysis and nucleic acid extraction can be obtained. Since the methods of the present disclosure are not necessarily limited to a specific organism or target, the methods do not require any functionalized surfaces or specialized reagents, resulting in significant savings, both in materials cost and required time. The methods described in the present disclosure can thus allow for a streamlined sample preparation workflow. After liberation of the nucleic acids, electric fields can be used for electrophoresis, capture and concentration of the nucleic acids.

In some embodiments, liberated nucleic acids can be eluted, for example into a receptacle similar to receptacle (315).

In another embodiment, electric fields can be used for both beadless lysis and nucleic acid capture. After cell lysis and liberation of the nucleic acids, electric fields can be used for electrophoresis, capture and concentration of the nucleic acids.

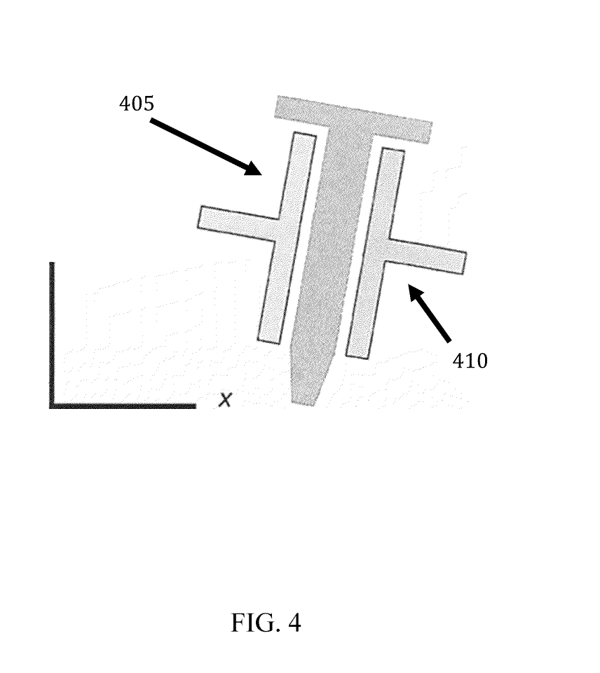

For example, referring to FIG. 4, time-varying electric fields can be applied across a centrifuge tube between two electrode (405, 410). The time-varying electric fields can be used to dissociate molecules, particles, and superstructures such as cell walls. By controlling the magnitude, frequency, and phase of the electric field between two contacts (405, 410), electrophoretic separation of ions can motivate cell lysis by either disintegration of cell walls and membranes or by osmosis.

Nucleic acids can then be captured as described in a previous embodiment. Supernatant, cell detritus, and unwanted products are discarded into a waste receptacle (such as element 315 in FIG. 3), while targets of interest are sequestered in the main chamber of the centrifuge tube (such as element 305 of FIG. 3).

One possible embodiment of the devices of the present disclosure comprises a standard centrifuge with added electrode pins mounted around the path of travel of the centrifuge.

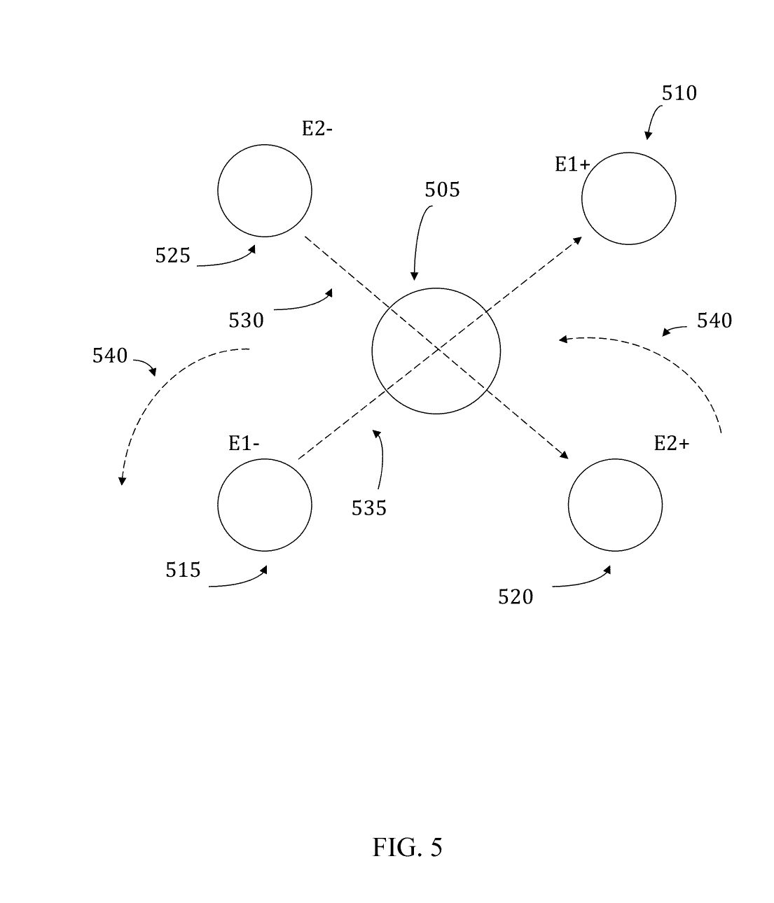

FIG. 5 illustrates an exemplary configuration where the centrifuge device has multiple electrodes, forming multiple electric fields, through which at least one centrifuge tube spins as the centrifuge device is operated.

In FIG. 5 multiple electrode dipole pairs are mounted around the travel path. The electrode pairs E1+ and E1− are denoted as elements (510) and (515) respectively. The centrifuge tube (505) is illustrated as crossing the electric field (535) in the direction of travel (540).

Electric field (535) is generated by electrodes (510) and (515) Electric field (530) is generated by electrodes (520) and (525).

Electrodes E1 (510, 515) and E2 (520, 525) are driven with, for example, ratiometric DC electric fields to allow quadrature vector control of the electric fields in the plane (535) and (530), as shown in FIG. 5. Electrodes E1 (510, 515) and E2 (520, 25) can also be driven with AC waveforms with a known phase offset between E1 and E2 to generate quadrature control of the AC waveform.

As understood by the persons skilled in the art, with two electrodes orthogonally mounted the electric fields can be generated in quadrature.

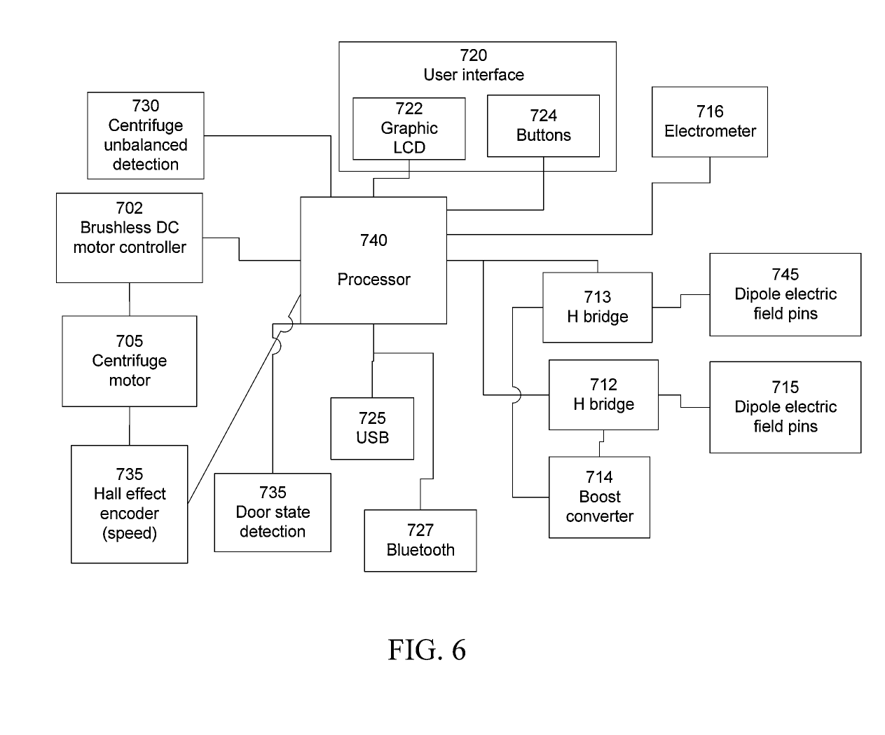

An example of control electronics for the centrifuge devices of the present disclosure implementation is shown in FIG. 6. A brushless DC motor is used as the centrifuge actuator. This motor is controlled using a standard BLOC motor controller and a hall-effect rotary encoder for speed feedback.

As illustrated in FIG. 6, in several embodiments of the disclosure, the main components of a centrifuge device comprise a Brushless DC (BLDC) motor controller (702) for spinning the centrifuge by controlling the centrifuge motor (705). In addition, this device has a user interface (720) to allow a user to monitor the device's status, as well as to allow the user to control what type of cycle to run.

Other components may comprise H bridges (712, 713) and a boost converter (714), as understood by the person skilled in the art.

As understood by the person skilled in the art, an H bridge is an electronic circuit that enables a voltage to be applied across a load in either direction. User interface (720) may comprise a display such as an LCD (722) and control input mechanisms, such as buttons (724).

Communication between the centrifuge device and a host computer may be implemented by USB (725), Bluetooth (727), or other communication protocols.

The communication interface (725, 727) relays information about the state of the device, and also may report information about the electric fields, rotational speeds, and any error conditions encountered during a run.

The centrifuge device may a variety of safety features to allow it to operate without damaging itself or the operator. For example, the centrifuge device may have a method of detecting if the centrifuge is unbalanced (730). Unbalanced centrifuges can shake violently, precess, and even ultimately cause injury or death. Circuit (730) can prevent the centrifuge device from running, if an out-of-balance condition is encountered.

Additionally, the centrifuge device may be equipped with a sensor (735) for determining the state of the centrifuge's lid. The centrifuge will not run if the lid is not securely closed.

A Hall effect encoder (735) can be used to regulate the speed of the motor (705).

A processor (740) may be used to regulate and control the different elements of a centrifuge device. An electrometer (716) may also be part of the centrifuge device, allowing a measurement on the electric fields generated by the electrodes in the centrifuge device.

A boost converter (714) or charge pump can be used to generate a high voltage DC {0-48VDC). Standard H-Bridge configurations can be used to modulate the electric field across the electric dipoles. By using a constant duty-cycle PWM waveform, the static electric field strength can be modulated as necessary. Using a time-varying PWM waveform a dynamic, AC electric fields can be generated as necessary.

The electrometer (716) provides a measurement of the electric field to confirm that the instrument is working. The electrometer (716) also provides feedback into the controller which regulates the generation of the electric fields.

Detection of an unbalanced centrifuge is primarily a safety function for the device. The unbalanced centrifuge detection system will use the same electrodes as those that generate the electric field to detect the presence or absence of necessary samples or counter-weights for the safe operation of the centrifuge.

Referring to FIG. 5, the capacitance between the electrode pairs E1+ (510) and E2− (525), and E2+ (520) and E1− (515) should measure the “open-air” capacitance between the two electrodes, with air as a dielectric. Measuring the capacitance between the two pin pairs E1+ (510) and E2+ (520), and E1− (515) and E2− (525) should provide the “sample” capacitance using the sample tube as the dielectric. If no change in capacitance is detected on the “sample” capacitance, then the system can safely assume that a particular centrifuge sample tube is empty. By comparing the waveforms generated using these capacitance measurements with the sensor output of the Hall-effect speed sensor, it can be determined if the centrifuge is improperly loaded.

A number of embodiments of the disclosure have been described. Nevertheless, it will be understood that various modifications may be made without departing from the spirit and scope of the present disclosure. Accordingly, other embodiments are within the scope of the following claims.

The examples set forth above are provided to those of ordinary skill in the art a complete disclosure and description of how to make and use the embodiments of the disclosure, and are not intended to limit the scope of what the inventor/inventors regard as their disclosure.

Modifications of the above-described modes for carrying out the methods and devices herein disclosed that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

It is to be understood that the disclosure is not limited to particular methods or devices, which can, of course, vary. It is also to be understood that the terminology used herein is for the purpose of describing particular embodiments only, and is not intended to be limiting. As used in this specification and the appended claims, the singular forms “a,” “an”, and “the ” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

What is claimed is:

1. A centrifuge device comprising:
a rotating head;
at least one centrifuge tube;
at least one slot in the rotating head, configured to accept the at least one centrifuge tube; and
at least one field element in the rotating head, wherein the at least one field element is configured to generate an electric field.

2. The centrifuge device of claim 1, wherein the at least one centrifuge tube has a filter configured to allow disposal of waste products, while retaining desired products.

3. The centrifuge device of claim 2, wherein the desired products comprise nucleic acids.

4. The centrifuge device of claim 2, further comprising at least one receptacle attached to a bottom end of the at least one centrifuge tube, wherein the at least one receptacle is for storing the waste products.

5. The centrifuge device of claim 4, wherein the at least one receptacle is for storing eluted nucleic acid products.

6. The centrifuge device of claim 1, wherein the at least one field element is a pair of electrodes.

7. The centrifuge device of claim 6, wherein the electric field direction is substantially perpendicular to an axial direction of the at least one centrifuge tube.

8. The centrifuge device of claim 1, wherein the electric field comprises two perpendicular components, the two perpendicular components lying in a plane perpendicular to an axial direction of the at least one centrifuge tube.

9. The centrifuge device of claim 1, wherein the at least one field element is fixed relative to the rotating head, and the at least one slot is configured to move through the magnetic field upon movement of the rotating head.

10. The centrifuge device of claim 1, further comprising a magnetic field element in the rotating head, wherein the magnetic field element is configured to generate a magnetic field.

11. A method comprising:
providing the centrifuge device of claim 1;
inserting a sample analyte in the at least one centrifuge tube, the sample analyte comprising cells; and
centrifuging the at least one centrifuge tube within the magnetic field, thereby causing shear forces between the cells, thereby lysing the cells, and thereby causing liberation of intracellular molecules.

12. The method of claim 11, further comprising disposing waste products into a first receptacle attached at a bottom end of the at least one centrifuge tube.

13. The method of claim 12, further comprising eluting the intracellular molecules into a second receptacle attached at a bottom end of the at least one centrifuge tube.

14. The method of claim 11, wherein the intracellular molecules are nucleic acids.
